(19) United States
(12) Patent Application Publication
(10) Pub. No.: US 2026/0171202 A1
(43) Pub. Date: Jun. 18, 2026

(54) INTRAVENOUS SET INTEGRATION INTO INFUSION INTEROPERABILITY WORKFLOW

(71) Applicant: CareFusion 303, Inc., San Diego, CA (US)

(72) Inventors: Adel SHAMS, Verona, NJ (US); Shangxian ZHU, Brookfield, WI (US)

(21) Appl. No.: 19/345,341

(22) Filed: Sep. 30, 2025

Related U.S. Application Data
(60) Provisional application No. 63/733,670, filed on Dec. 13, 2024.

(51) Int. Cl. G16H 10/60; A61M 5/142; G06K 7/14

(52) U.S. Cl. CPC G16H 10/60; A61M 5/142; A61M 2205/502; A61M 2205/6072; G06K 7/1413

(57) ABSTRACT

Methods are provided that include scanning, by a scanner, a barcode of an intravenous (IV) set, placing the IV set into service and automatically populating, by one or more processors in response to the scanning of the IV set barcode, fields of an electronic medical record system associated with a patient or fields of an infusion pump display. Systems and non-transitory machine-readable storage medium are also provided.

### CROSS-REFERENCE TO RELATED APPLICATION

The present application claims priority under 35 U.S.C. § 119 to U.S. Provisional Ser. No. 63/733,670 , entitled “INTRAVENOUS SET INTEGRATION INTO INFUSION INTEROPERABILITY WORKFLOW,” filed on Dec. 13, 2024, the entirety of which is incorporated herein by reference.

### TECHNICAL FIELD

This application relates generally to intravenous (IV) tubing or IV set management and more particularly to an automated system to improve the accuracy and reliability of IV tubing or IV set management while seamlessly integrating with existing hospital infrastructure and healthcare professionals'workflows.

### BACKGROUND

IV therapy plays a crucial role in healthcare, allowing for the delivery of medications, fluids and nutrients directly into the patient's bloodstream. This method is often administered through infusion pumps, which provide controlled and accurate delivery of medications. A key component of this system is the IV tubing, which must be properly chosen, labeled and changed at appropriate intervals to ensure patient safety and prevent complications, such as infections or incorrect medication dosing. Critically ill patients, such as in the intensive care unit (ICU) or the operating room (OR), commonly receive multiple high-alert IV infusions simultaneously (e.g., 6-8 IV tubing).

### SUMMARY

According to various aspects, the subject technology provides a method including scanning, by a scanner, a barcode of an intravenous (IV) set; placing the IV set into service; and automatically populating, by one or more processors in response to the scanning of the IV set barcode, any of: one or more fields of an electronic medical record system associated with a patient; one or more fields of an infusion pump display; a notification indicator; and a reminder indicator.

According to various aspects, the subject technology provides a system having one or more processors and memory including instructions that, when executed by the one or more processors, cause the system to: receive information from a scanned barcode of an intravenous (IV) set; and automatically populate, based on the information from the scanned barcode, any of: one or more fields of an electronic medical record system associated with a patient; one or more fields of an infusion pump display; a notification indicator; and a reminder indicator.

According to various aspects, the subject technology provides a non-transitory machine-readable storage medium embodying instructions that, when executed by a machine, cause the machine to perform operations comprising: receiving scanned barcode information from an intravenous (IV) set; automatically populating, based on the scanned barcode information from the IV set, one or more fields of an electronic medical record system associated with a patient; receiving scanned barcode information from an identification tag on the patient; receiving scanned barcode information from a fluid container; receiving scanned barcode information from an infusion pump; and associating the IV set, the fluid container and the infusion pump with the patient in the electronic medical record system.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example of an institutional patient care system of a healthcare organization, according to aspects of the subject technology.

FIG. 2 depicts an example of infusion interoperability workflow system, in accordance with aspects of the subject technology.

FIG. 3 depicts an example data field structure for a barcode of an IV set, according to aspects of the subject technology.

FIG. 4 is a conceptual diagram illustrating an example electronic system for an infusion interoperability workflow system, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

One of the major concerns in IV therapy is the management of IV tubing, specifically, ensuring that IV tubing is changed at appropriate intervals to prevent complications like infection, occlusion or medication dosing errors. Currently, nurses are required to place physical sticker labels on IV tubing, which display important information such as the change-out date and time. Additionally, nursing staff must manually document the IV tubing details in the patient's electronic medical record (EMR). This includes information like the type of IV tubing (e.g., primary or secondary), the time and date of the last tubing change, whether new IV tubing is used and any other relevant notes.

However, the existing system of using IV tubing sticker labels has several issues. One issue is human error in that nurses may place incorrect or incomplete information on the labels or even forget to label the line entirely. This can result in potential errors in tubing change schedules or medication administration.

Another issue is label misplacement where physical labels may become dislodged, smudged or become difficult to read due to the clinical environment.

Yet another issue is lack of accountability in IV tubing change documentation. In current systems there is limited accountability for ensuring that IV tubing is changed at the appropriate intervals. When nursing staff are busy or under pressure, they may inadvertently tear off labels or create new ones without accurately recording the previous change-out time. This can lead to discrepancies in documentation, which increases the risk of tubing being used beyond the recommended period and potentially compromising patient safety.

Another issue is inconsistent documentation in that manual entry into the EMR increases the chances of inconsistencies between the label on the tubing and the information in the patient's medical records.

A further issue is the potential for infection control violations in that when labels are incorrect or missing, nurses may inadvertently use IV tubing beyond its safe time frame, increasing the risk of infections like a catheter-related bloodstream infection (CRBSI) or a central line-associated bloodstream infection (CLABSI).

The subject technology includes a system that seamlessly integrates IV sets with infusion pumps and supports EMR interoperability workflows. According to some implementations, each IV set may include a 2D or 1D barcode, depending on the available infrastructure of the hospital or health care facility, positioned along the IV tubing (e.g., below the drip chamber). As part of the IV infusion interoperability workflow, healthcare professionals may be required to scan the IV set barcode, along with the patient ID, medication and infusion pump barcode each time they start an infusion.

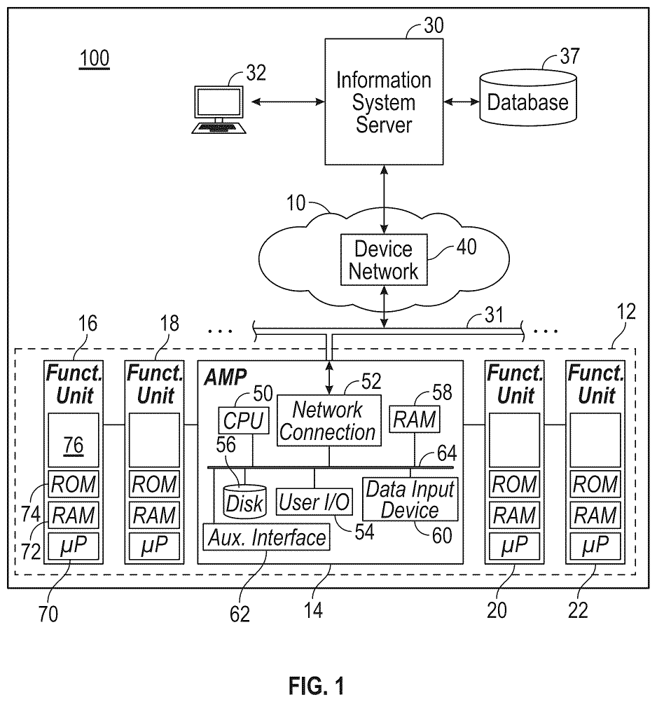

FIG. 1 depicts an example of an institutional patient care system 100 of a healthcare organization, according to aspects of the subject technology. In FIG. 1, a patient care device (or “medical device” generally) 12 is connected to a healthcare facility network 10. The term patient care device (PCD) may be used interchangeably with the term patient care unit (PCU), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each medical device 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a healthcare facility. For example, network 10 of FIG. 1 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 40 by which patient care devices 12 (and other devices) communicate in accordance with normal operations.

Additionally, institutional patient care system 100 may incorporate a separate information system server 30, the function of which will be described in more detail below. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistances, mobile devices such as laptops, tablet computers, augmented reality devices, or smartphones, configured with software for communications with information system server 30 via network 10.

Patient care device 12 comprises a system for providing patient care, such as that described in U.S. Pat. No. 5,713,856 to Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control module or unit 14, also referred to as interface unit 14, connected to one or more functional modules 16, 18, 20, 22. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally, or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the data input device 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device.

Although data input device 60 is shown in FIG. 1 to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with control unit 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 16, 18, 20, 22 are any devices for providing care to a patient or for monitoring patient condition. As shown in FIG. 1, at least one of functional modules 16, 18, 20, 22 may be an infusion pump module such as an intravenous infusion pump for delivering medication or other fluid to a patient. For the purposes of this discussion, functional module 16 is an infusion pump module. Each of functional modules 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor or an intracranial pressure monitor or the like. Functional module 18, 20 and/or 22 may be a printer, scanner, bar code reader or any other peripheral input, output or input/output device.

Each functional module 16, 18, 20, 22 communicates directly or indirectly with interface unit 14, with interface unit 14 providing overall monitoring and control of device 12. Functional modules 16, 18, 20, 22 may be connected physically and electronically in serial fashion to one or both ends of interface unit 14 as shown in FIG. 1, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1, any number of devices may be connected directly or indirectly to central control unit 14. The number and type of functional modules described herein are intended to be illustrative, and in no way limit the scope of the subject technology. Module-specific components 76 include any components necessary for operation of a particular module, such as a pumping mechanism for infusion pump module 16. While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Patient care device 12 is capable of operating in several different modes, or personalities, with each personality defined by a configuration database. The configuration database may be a database on storage unit 56 internal to patient care device 12, or an external database 37. A particular configuration database is selected based, at least in part, by patient-specific information such as device or patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 12 location in the hospital or hospital computer network. Patient care information may be entered through network connection 52 or any of input/interface devices 54, 60 or 62, and may originate from anywhere in network 10, such as, for example, from a pharmacy server, admissions server, laboratory server, and the like.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction or a combination of both automated and manual interaction. Automated interaction may be continuous or intermittent and may occur through direct network connection (as shown in FIG. 1), or through RS232 links, MIB systems, RF links such as BLUETOOTH, IR links, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in HIS server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs and maintain open communication.

A medical device including one or more of the features described may be implemented an ambulatory medical device. Ambulatory medical devices generally refer to devices designed to be portable to support administration of medication during transportation of a patient or remote medication administration (e.g., outside of a health care facility such as in a user's home). U.S. Pat. No. US7,163,381 to Barak describes a pump that may be suitable for ambulatory care and modified to include the IV tubing management features described to assist in providing safe administration during mobility events. The disclosure of U.S. Pat. No. 7,163,381 is incorporated by reference in its entirety.

As discussed above, the subject technology includes a system that seamlessly integrates IV sets with infusion pumps and supports EMR interoperability workflows. Thus, the disclosed system leverages barcodes on IV sets to encode essential data for EMR documentation. Each barcode can include any of 1) unique IV tubing identifiers to ensure each IV tubing is identifiable, 2) filter types to document whether the IV set includes a filter or not and if a filter is present to specify its type (e.g. 0.2, 1.2 μm), 3) tubing types to document the specific types of tubing used (e.g. microbore, smallbore) and/or documenting tubing material (e.g. PVC, TPE), 4) indicating whether the tubing is a primary or secondary IV tubing and 5) capturing any other desired or necessary details for comprehensive IV tubing documentation, such as type of drip chamber, tubing light resistance, etc.

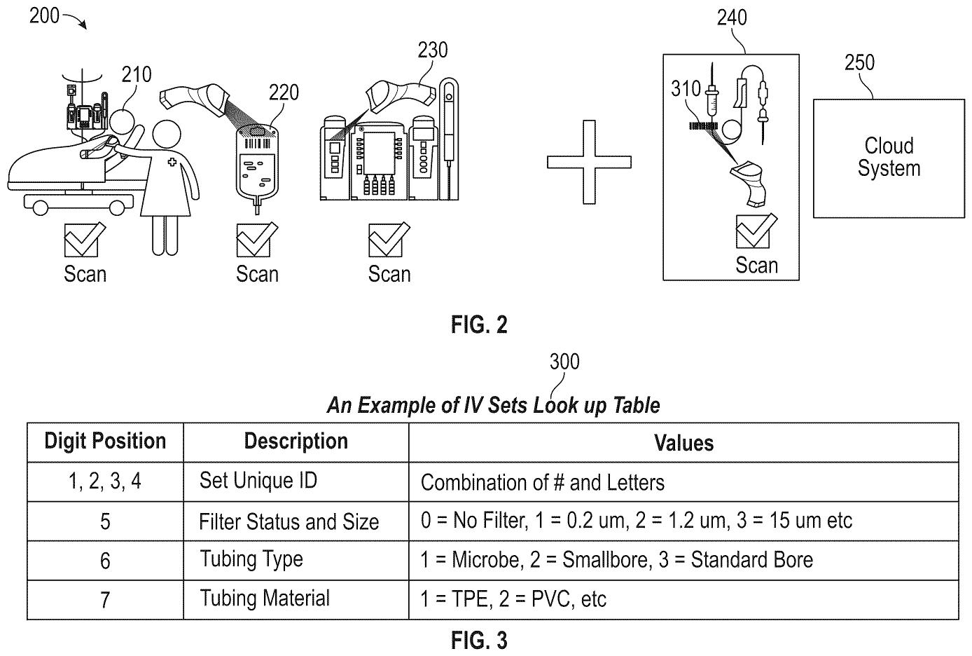

FIG. 2 depicts an example of an infusion interoperability workflow system 200 of a healthcare organization, according to aspects of the subject technology. In FIG. 2, an infusion process includes scanning (e.g., by a health care professional) a patient's identification (e.g., identification wristband) 210, scanning a barcode on a fluid container (e.g., IV bag, medication bag) 220, scanning a barcode on an infusion pump 230 and scanning an IV set 310 barcode 240 that is put into service for an infusion process between the fluid container and the patient. Any of the information may be provided to a cloud system 250.

FIG. 3 depicts an example of data field structure 300 for a barcode of an IV set 310 (e.g., IV set look-up table). Once the barcode of the IV set 310 is scanned, the related information can be automatically populated into the corresponding EMR fields of the system 200 and/or provided to a cloud system 250 having various software platforms (e.g., drug library).

This information provided by the barcode may then be used to auto populate the EMR field related to the line change. For example, a unique IV set ID allows the system 200 to recognize when a new IV set 310 is used. If it is a new IV set 310, the answer to whether IV tubing has changed will be “yes.” Otherwise, if the IV set ID was the same as that of the previous infusion, then the system 200 shall log in with “no” as an answer to whether IV tubing has changed. The date and time when the new IV set ID is identified may then be used to populate the fields “date tubing changed” and “tubing changing time.” Secondary or primary line from the system may be used to populate the field “tubing type.”

According to various implementations, automatically documenting the IV set 310 change-out date and time allows the system 200 to send alerts (e.g., remind healthcare providers of the upcoming change-out time) and track IV set 310 change-out (e.g., display and track IV set 310 change-out information on multiple platforms, such as IV infusion pumps, EMR patient dashboards, user task list or other dashboards for easy monitoring).

During an auto programming request (APR) scanning process, the system 200 and/or the cloud system 250 automatically records the timestamp of the infusion start when an infusion starts. By scanning of the IV set 310 during the APR workflow, the unique ID of the IV set 310 may be used to determine which IV set 310 is being used. Infusion time associated with this particular IV set 310 may be calculated on an accumulative basis. When the accumulative time reaches the change out period, a reminder may be generated. The change out time is a programmable input according to the protocol of the facility. The change-out date/time may be automatically added to a healthcare professionals'task list in the EMR, informing/reminding them of the IV set 310 change-out date.

According to various implementations, the system 200 may enhance patient safety by simultaneously integrating drug incompatibility detection and line compatibility checks into the APR workflow at the point of care. For example, by scanning the barcode on the IV set 310, the system 200 and/or the cloud system 250 (e.g., drug library) can identify the specific infusion running inside the IV set 310 since the system 200 is aware of the current medication being administered through APR workflow.

Also, when new medication is added to an IV line of the IV set 310 (e.g. access port or as a secondary bag), healthcare professionals can input the name of the new medication into the system 200 or scan a new medication label generated at a pharmacy or a dispensing system. This step ensures that the system 200 has up-to-date information on all medications being administered through the IV set 310.

In addition, the system 200 and/or the cloud system 250 may include a comprehensive library of drug interactions and incompatibilities. This library may be continuously updated to reflect the latest medical knowledge and guidelines. Upon scanning the barcode and entering the new medication, the system 200 may cross-reference the current infusion with the drug interaction library. If any potential drug interactions or line incompatibilities are detected, the system 200 may immediately alert healthcare providers.

According to various implementations, by capturing data provided by the barcode of the IV set 310, the system 200 will have real clinical and operational insights into IV set 310 usage and provide actional analytics to guide IV tubing selection and optimization of IV tubing inventory management.

For example, the system 200 may ensure the correct IV set 310 is used for a particular patient and a particular medication. Here, the system 200 and/or the cloud system 250 may provide advanced analytics and reporting tools to identify the proper IV set 310 was chosen based on patient and medication profiles and/or infusion data from a pump system (e.g., hardware, software) and IV sets 310. As another example, the system 200 may provide supply chain management by tracking usage of IV sets 310 retrospectively to inform procurement decisions. In another example, the system 200 may provide compliance and audit trails by ensuring all actions with IV sets 310 are logged with timestamps and user identification to maintain compliance with regulatory requirements and to facilitate audits.

According to various implementations, the system 200 may automatically document IV set 310 information in the EMR. Automated documentation by the system 200 may be essential to accurately capture and record important details, such as the tubing type, change-out time, and connection duration, directly into the EMR. This may eliminate the risk of human error, ensure consistency, and enhance the overall accuracy of patient records, improving both patient safety and compliance with healthcare protocols.

According to various implementations, the system 200 may provide real-time reminders. Automated reminders may be sent directly to nursing staff via infusion pumps or EMR systems, ensuring that IV set 310 change-outs occur on schedule. These reminders may help prevent errors associated with missed or forgotten changes, thus reducing the risk of infection and improving patient outcomes.

According to various implementations, the system 200 and/or the cloud system 250 may enable efficient resource allocation. By having a system 200 that tracks and predicts IV set 310 change-out times, nursing teams can better budget their time and resources, ensuring that staffing levels are appropriate to meet patient care needs. This would enable more effective task planning and reduce unnecessary strain on healthcare workers.

According to various implementations, the system 200 and/or the cloud system 250 may provide for digital twins in IV set 310 or IV tubing management. Digital twin technology, which creates a real-time virtual representation of physical systems, can play a transformative role in IV set 310 management. By utilizing digital twins, healthcare providers can simulate and monitor IV therapy processes, including IV set 310 change-out schedules and medication administration. This technology provides real-time insights into the condition and usage of IV sets 310, allowing for predictive maintenance and ensuring timely replacements. Digital twins can simplify auditing and help ensure hospital compliance with IV set 310 change-out protocols, eliminating the need for manual audits or hiring third-party enterprise software companies for auditing.

Computer program code for carrying out operations of the subject technology may be written in an object oriented programming language such as, for example, JAVA®, Smalltalk, or C++. However, the computer program code for carrying out operations of the subject technology may also be written in conventional procedural programming languages, such as the “C” programming language, in an interpreted scripting language, such as Perl, or in a functional (or fourth generation) programming language such as Lisp, SML, Forth, or the like. The software may also be written to be compatible with HLA-7 requirements.

Many steps of the above-described system 100, 200, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

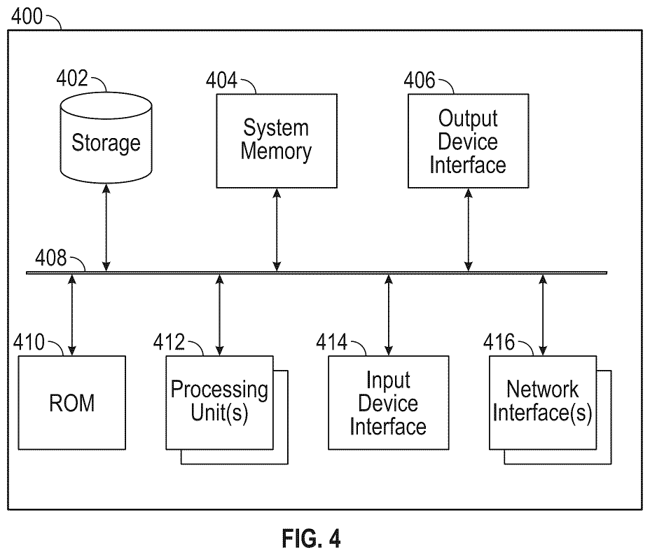

FIG. 4 is a conceptual diagram illustrating an example electronic system 400 for automatically adapting control of a medical device responsive to detecting a location setting change, according to aspects of the subject technology. Electronic system 400 may be a computing device for execution of software associated with one or more portions or steps of system 100, 200, or components and processes provided by FIGS. 1-3, including but not limited to information system server 30, device terminal 32, computing hardware within patient care device 12, or external database 37. Electronic system 400 may be representative, in combination with the disclosure regarding FIGS. 1-3. In this regard, electronic system 400 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 400 may include various types of computer readable media and interfaces for various other types of computer readable media. In the depicted example, electronic system 400 includes a bus 408, processing unit(s) 412, a system memory 404, a read-only memory (ROM) 410, a permanent storage device 402, an input device interface 614, an output device interface 406, and one or more network interfaces 416. In some implementations, electronic system 400 may include or be integrated with other computing devices or circuitry for operation of the various components and processes previously described.

Bus 408 collectively represents all system, peripheral, and chipset buses that communicatively connect the numerous internal devices of electronic system 400. For instance, bus 408 communicatively connects processing unit(s) 412 with ROM 410, system memory 404, and permanent storage device 402.

From these various memory units, processing unit(s) 412 retrieves instructions to execute and data to process in order to execute the processes of the subject disclosure. The processing unit(s) can be a single processor or a multi-core processor in different implementations.

ROM 410 stores static data and instructions that are needed by processing unit(s) 412 and other modules of the electronic system. Permanent storage device 402, on the other hand, is a read-and-write memory device. This device is a non-volatile memory unit that stores instructions and data even when electronic system 400 is off. Some implementations of the subject disclosure use a mass-storage device (such as a magnetic or optical disk and its corresponding disk drive) as permanent storage device 402.

Other implementations use a removable storage device (such as a floppy disk, flash drive, and its corresponding disk drive) as permanent storage device 402. Like permanent storage device 402, system memory 404 is a read-and-write memory device. However, unlike storage device 402, system memory 404 is a volatile read-and-write memory, such a random access memory. System memory 404 stores some of the instructions and data that the processor needs at runtime. In some implementations, the processes of the subject disclosure are stored in system memory 404, permanent storage device 402, and/or ROM 410. From these various memory units, processing unit(s) 412 retrieves instructions to execute and data to process in order to execute the processes of some implementations.

Bus 408 also connects to input and output device interfaces 414 and 406. Input device interface 414 enables the user to communicate information and select commands to the electronic system. Input devices used with input device interface 414 include, e.g., alphanumeric keyboards and pointing devices (also called “cursor control devices”). Output device interfaces 406 enables, e.g., the display of images generated by the electronic system 400. Output devices used with output device interface 406 include, e.g., printers and display devices, such as cathode ray tubes (CRT) or liquid crystal displays (LCD). Some implementations include devices such as a touchscreen that functions as both input and output devices.

Also, as shown in FIG. 4, bus 408 also couples electronic system 400 to a network (not shown) through network interfaces 416. Network interfaces 416 may include, e.g., a wireless access point (e.g., Bluetooth or WiFi) or radio circuitry for connecting to a wireless access point. Network interfaces 416 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 400 can be used in conjunction with the subject disclosure.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

The functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device (e.g., a mouse or a trackball), by which the user can provide input to the computer. Other kinds of devices can be used to provide for interaction with a user as well. For example, information can be displayed to the user on an infusion pump (e.g., LCD display). As another example, feedback provided to the user can be any form of sensory feedback (e.g., visual feedback, auditory feedback, or tactile feedback) and input from the user can be received in any form (e.g., acoustic, speech, or tactile input). In addition, a computer can interact with a user by sending documents to and receiving documents from a device that is used by the user (e.g., by sending web pages to a web browser on a user's client device in response to requests received from the web browser).

Embodiments of the subject matter described in this specification can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the subject matter described in this specification), or any combination of one or more such back end, middleware, or front end components. The components of the system can be interconnected by any form or medium of digital data communication (e.g., a communication network). Examples of communication networks include a local area network (LAN) and a wide area network (WAN), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein but are to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms web page and server. The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. A method, comprising:
scanning, by a scanner, a barcode of an intravenous (IV) set;
placing the IV set into service; and
automatically populating, by one or more processors in response to the scanning of the IV set barcode, any of:
one or more fields of an electronic medical record system associated with a patient;
one or more fields of an infusion pump display;
a notification indicator; and
a reminder indicator.

2. The method of claim 1, further comprising:
scanning, by the scanner, an identification tag on the patient; and
associating the IV set with patient information in the electronic medical record system.

3. The method of claim 1, further comprising:
scanning, by the scanner, a barcode of a fluid container; and
associating the fluid container with patient information in the electronic medical record system.

4. The method of claim 1, further comprising:
scanning, by the scanner, a barcode of an infusion pump; and
associating the infusion pump with patient information in the electronic medical record system.

5. The method of claim 1, wherein the automatically populating the one or more fields of the electronic medical record system comprises indicating one of:
the IV set is being used for an initial process with the patient;
the IV set is being reused for an additional process with the patient; and
the IV set is replacing a previous IV set connected to the patient.

6. The method of claim 1, wherein the automatically populating the one or more fields of the electronic medical record system comprises a date and time when the IV set was scanned.

7. The method of claim 1, wherein the automatically populating the one or more fields of the electronic medical record system comprises whether the IV set is a primary line or a secondary line.

8. The method of claim 1, further comprising:
starting a timer associated with the IV set; and
generating, by the one or more processors, an alert that the IV set has reached its change-out date and time.

9. The method of claim 1, further comprising:
starting a timer associated with the IV set; and
automatically populating, by the one or more processors, a change-out date and time for the IV set on a healthcare professionals'task list in the electronic medical record system.

10. The method of claim 1, further comprising:
identifying, by the one or more processors, a current infusion solution running inside the IV set based on the scanning of the IV set barcode.

11. The method of claim 10, further comprising:
inputting, by a health care professional into the electronic medical record system, an additional medication to be added to the IV set through one of an access port of the IV set and a secondary fluid container.

12. The method of claim 11, further comprising:
cross-referencing, by the one or more processors, the current infusion solution and the additional medication within a drug interaction database.

13. The method of claim 12, further comprising:
generating, by the one or more processors, an alert that the current infusion solution and the additional medication will have a negative drug interaction.

14. The method of claim 12, further comprising:
generating, by the one or more processors, an alert that one of the current infusion solution, a drug-line feature and the additional medication are incompatible with the IV set.

15. A system, comprising:
one or more processors; and
memory including instructions that, when executed by the one or more processors, cause the system to:
receive information from a scanned barcode of an intravenous (IV) set; and
automatically populate, based on the information from the scanned barcode, any of:
one or more fields of an electronic medical record system associated with
a patient;
one or more fields of an infusion pump display;
a notification indicator; and
a reminder indicator.

16. The system of claim 15, wherein the instructions further cause the system to:
receive information from a scanned barcode of an identification tag on the patient;
receive information from a scanned barcode of a fluid container;
receive information from a scanned barcode of an infusion pump; and
associating the IV set, the fluid container and the infusion pump with the patient in the electronic medical record system.

17. The system of claim 15, wherein the instructions further cause the system to determine one of:
the IV set is being used for an initial process with the patient;
the IV set is being reused for an additional process with the patient; and
the IV set is replacing a previous IV set associated with the patient.

18. The system of claim 15, wherein the instructions further cause the system to:
start a timer associated with the IV set; and
one of generate an alert that the IV set has reached its change-out date and time and automatically populate the change-out date and time for the IV set on a healthcare professionals'task list in the electronic medical record system.

19. The system of claim 15, wherein the instructions further cause the system to:
identify a current infusion solution running inside the IV set based on the scanning of the IV set barcode;
receive an additional medication to be added to the IV set;
cross-reference the current infusion solution and the additional medication within a drug interaction database; and
generate an alert that one of:
the current infusion solution and the additional medication will have a negative drug interaction; and
one of the current infusion solution and the additional medication are incompatible with the IV set.

20. A non-transitory machine-readable storage medium embodying instructions that, when executed by a machine, cause the machine to perform operations comprising:
receiving scanned barcode information from an intravenous (IV) set;
automatically populating, based on the scanned barcode information from the IV set, one or more fields of an electronic medical record system associated with a patient;
receiving scanned barcode information from an identification tag on the patient;
receiving scanned barcode information from a fluid container;
receiving scanned barcode information from an infusion pump; and
associating the IV set, the fluid container and the infusion pump with the patient in the electronic medical record system.
